(19) United States
(12) Patent Application Publication
(10) Pub. No.: US 2024/0416101 A1
(43) Pub. Date: Dec. 19, 2024

(54) COUPLING SYSTEM FOR FLUID TRANSFER

(71) Applicant: Interlinked AB, Stockholm (SE)

(72) Inventors: Robert Axelsson, GRÄNNA (SE); Katarina Hedbeck, Stockholm (SE); Rebecca Bejhed, Uppsala (SE); Christopher Blacker, Uppsala (SE)

(73) Assignee: Interlinked AB, Stockholm (SE)

(21) Appl. No.: 18/716,499

(22) PCT Filed: Dec. 5, 2022
(86) PCT No.: PCT/EP2022/084458

§ 371 Date: Jun. 4, 2024

(30) Foreign Application Priority Data
Dec. 6, 2021 (SE) 2151482-3

(51) Int. Cl. A61M 39/10

(52) U.S. Cl. CPC A61M 39/105; A61M 39/1011

(57) ABSTRACT

A coupling system for fluid transfer and a system for transferring fluids into a patient are provided. The coupling system comprising a first tube, at least one second tube, a coupling device configured for a connection of the at least one second tube with the first tube, wherein the coupling device comprises at least one first connector element and at least one second connector element configured to matingly connect for a connection between the at least one second tube and the first tube, wherein the first connector element comprises a first sealing element and a tube portion, and wherein the second connector element comprises a second sealing element, whereby in a connected state of the coupling device, the tube portion projects through the first and second sealing elements for enabling a transfer of fluid through the coupling device.

### FIELD OF THE INVENTION

The present invention generally relates to the field of medical devices. More specifically, the present invention relates to a system for the introduction of fluids and particularly to a system for delivering medications by intravenous, intraarterial, intraosseous, intrathecal, epidural or similar introduction.

### BACKGROUND OF THE INVENTION

It is common practice to administer medications to patients in fluid form through, for example, intravenous (IV) introduction. Such infusions are customarily carried out by employing IV kits that incorporate one or more supply bottles or bags, or other containers of liquid that are suspended above a patient.

IV kits may comprise a “primary line”, wherein gravity or an infusion pump causes liquid to flow through a tube to a venous access device (VAD) which is implanted in the patient, typically in a peripheral vein in the arm or in a central vein closer to the heart. Many treatments require that a patient receive, sometimes over an extended period of several hours, a fluid such as a saline solution or a dextrose solution. These fluids (collectively referred to as “primary fluids” herein) are used to treat a number of symptoms that present in a wide variety of medical conditions-symptoms such as dehydration or hypoglycemia.

A “secondary line”, used for continuous or intermittent drug infusion, may comprise one or more containers, tubings, as well as a microdrip or macrodrip system, connected to the lower Y port (secondary port) of the primary line instead of to a IV catheter. When used continuously, the secondary (IV) line permits drug infusion and titration while the primary line maintains a constant total infusion rate. When used intermittently, the secondary line is commonly called a piggyback set. In this case, the primary line maintains venous access between drug doses. Typically, a piggyback set includes a small IV container, short tubing, and a macrodrip system. This set connects to the primary line's upper Y port, also called a piggyback port. Antibiotics are most commonly administered by intermittent (piggyback) infusion. To make this set work, the primary IV container is positioned below the piggyback container. The primary fluid is thereby hindered to flow during the drug infusion via the secondary line. After administration of the drug(s), primary fluid is allowed to flow through the primary line such that all drug content is provided to the patient.

According to today's methodology, the secondary line is most commonly coupled or attached to the primary line by means of needleless connectors (NCs) which are integrated with the primary line. The NCs are devices which connect to the end of vascular catheters and enable catheter access for infusion and aspiration, and the NCs have several possible design characteristics which determines how they should operate and be operated. However, as there are various different designs of the NCs, this makes it difficult for medical staff to identify the features which present the least risk and greatest safety. Furthermore, the designated clamping-disconnection sequence is of vital importance, as this is required for the NCs to work as designed. More specifically, NCs with a negative fluid displacement should be clamped before disconnection and positive fluid displacement NCs clamped after disconnection, as clamping is required to prevent air embolism. Also, it may be required of the medical institution (e.g. hospital) that a closed system transfer device (“CSTD”) is used, wherein the CSTD device mechanically prohibits the transfer of environmental contaminants into a system and the escape of hazardous drug or vapor concentrations outside the system. Hence, CSTD devices aims to prevent an uncontrolled inflow and outflow of contaminants and drugs, preserving the quality of solution to be infused into a patient, and the CSTD devices have been developed to prevent occupational exposure to hazardous drugs and to protect patients, healthcare personnel and the environment from their effects. For the IV kit as described, the CSTD device may comprise two parts of which a first part is arranged on the primary line and the second part is arranged on the secondary line. However, CSTD devices may present challenges to medical staff, due to lack of standards, guidance and/or requirements. Furthermore, as there are numerous variabilities on the functionality of CSTD devices, the efficacy of these depends on their designs, which may be problematic.

Yet another problem is related to the risk of leakage of couplings and/or components of IV kits according to the prior art, as it is desirable that any leakage of fluid is mitigated. For example, if the infusion liquid is toxic, any leakage from a broken coupling, component, tube, etc., may be especially hazardous.

Hence, it is desired to provide alternatives to prior art arrangements for the administration of medications to patients in fluid form. More specifically, there is a wish to avoid the problems and difficulties related to the different operations, designs and functions of the NCs, to overcome the risks and/or problems related to the clamping associated with the NCs and/or to overcome or at least reduce the challenges related to CSTD devices. Furthermore, it is desired to provide reliability in terms of sealing and anti-leakage for components and/or systems for medication administration.

### SUMMARY OF THE INVENTION

It is an object of the present invention to mitigate one or more of the above problems and to provide a system for the introduction of fluids to a patient by intravenous, intraarterial, intraosseous, intrathecal, epidural or similar introduction which may mitigate at least some risks, problems and/or complications related to NCs, clamping operations and/or CSTDs devices, whilst being able to provide excellent sealing and anti-leakage properties.

This and other objects are achieved by providing a system having the features in the independent claim. Preferred embodiments are defined in the dependent claims.

Hence, according to the present invention, there is provided a coupling system for fluid transfer. The coupling system comprises a first tube arranged to transfer a first fluid, at least one second tube arranged to transfer a respective second fluid, and a coupling device configured for a connection of the at least one second tube with the first tube. The coupling device comprises a manifold structure comprising at least one first connector element and at least one second connector element configured to matingly connect for a connection between the at least one second tube and the first tube for a transfer of the respective second fluid from the at least one second tube to the first tube, wherein the first connector element comprises a first sealing element and a tube portion, and wherein the second connector element comprises a second sealing element. In a non-connected state of the coupling device, the first sealing element and the second sealing elements seal the respective second tube from the first tube. In a connected state of the coupling device, the tube portion projects through the first and second sealing elements, for fluid connection between the tube portion of the first connector element and the second connector element for enabling a transfer of fluid through the coupling device.

Thus, the present invention is based on the idea of providing a coupling system which is able to provide a connection of one or more (second) tube(s) to a (first) tube via a coupling device having a manifold construction by its one or more first and second connector elements. The coupling system may hereby enable a convenient, easy, intuitive coupling operation. The coupling system may enable a transfer of fluid through the coupling device which is sealed to the outside environment when the coupling device is connected, and a leak-free interruption of a transfer of (a) fluid(s) in case the coupling device is disconnected. More specifically, upon connection of the coupling device, a transfer of fluid is enabled by the tube portion of the coupling device, projecting through (penetrating) the first and second sealing elements (e.g. via (by) a closed pointed tip of the end portion of the tube portion) for fluid transfer through the coupling device.

The coupling system of the present invention is advantageous in that it mitigates at least some risks, problems and/or complications related to NCs, clamping operations and/or CSTDs devices, whilst being able to provide excellent sealing and anti-leakage properties. By its uncomplicated configuration or structure, the coupling system is conveniently arranged and used. Notably, the coupling system may be construed as being a CSTD device in itself by the integrated coupling device and tubes.

The coupling system of the present invention is advantageous in that its connection and reconnection (if detached or disconnected) by the coupling device is performed in an easy, convenient and efficient manner. Notably, the first and second connector elements of the coupling device are connectable via an easy and convenient plug operation, which may not need any screwing or twisting. For example, in case a tube connected to a coupling device according to the coupling system of the present invention is pulled apart, it may be desirable to be able to reinstate the (infusion) therapy again as quickly as possible. The coupling device of the coupling system meets this demand, as the (re) connection of the coupling device may be performed quickly and intuitively due to the innovative configuration of the coupling device.

It will be appreciated that the coupling device of the coupling system may be disconnected by an operation which is reversed to the connection operation as described above. Hence, the tube portion, which during connection of the coupling device projects through the first and second sealing elements, may be retracted (pulled), in which the first and second sealing elements are configured to abut each other and sealingly separate the first tube and the respective second tube. Ultimately, in a (completely) disconnected state of the coupling device, the first and second connector elements become separated from each other.

Consequently, after the disconnection of the coupling device, the coupling device subsequently becomes sealed again by the first and second sealing elements. In other words, the respective first and second sealing elements may conveniently stop a flow on either side of the respective sealing elements upon disconnection/detachment of the coupling device. Hence, the coupling device of the present invention may provide excellent sealing properties and avoid leakage, both during a transfer of fluid through the coupling device as well as during an interrupted flow of fluid through the coupling device as a result of a detachment or disconnection of the coupling device.

The coupling device of the present invention is particularly advantageous by the features of the tube portion. For example, the tube portion may comprise a closed pointed tip at the end portion of the tube portion, as the tube portion thereby conveniently and easily may penetrate the first and/or second sealing elements upon connection of the coupling device. More specifically, the pointed tip is configured to decrease the friction between the tube portion and the first and/or second sealing elements, which consequently leads to an even further convenient connection and/or disconnection of the coupling device. Hence, compared to a relatively blunt end portion of a tube, the coupling device of the present invention is able to achieve a more convenient connection and/or disconnection operation of the coupling device.

It will be appreciated that the ability of the coupling system to mitigate any leakage of fluid is advantageous for reasons of safety. For example, in case the coupling system is provided for a transfer of a toxic liquid, any leakage from the coupling system may be particularly hazardous. Hence, the coupling system of the present invention may significantly increase the safety of medical staff and/or patients.

Furthermore, the coupling system of the present invention is advantageous in that it may save fluid by its advantageous sealing properties. For example, in case the coupling system is used for a transfer of blood, the coupling system may mitigate any loss of blood and/or a contamination of the environment caused by a leakage in case of a detachment of the coupling device. Furthermore, by its excellent sealing properties, the coupling device of the coupling system may mitigate any contamination of the fluid (e.g. blood) transferred through the coupling system.

The present invention is further advantageous in that the coupling system provides a convenient detachment (disconnection) of the coupling device. For example, a pulling of the first and/or second tubes, e.g. by a patient and/or medical staff, may detach the first and second connector elements of the coupling device from each other, thereby mitigating any further pulling of the tube(s) at the other end thereof. It will be appreciated that it may be desirable that (medical) tubes commonly used at hospitals, nursing homes, clinics, etc., are equipped with a coupling system of the present invention, as the coupling device of the coupling system may constitute “weak link(s)” of the tube(s). Hence, if one or more tubes is (are) connected between a patient and a source (e.g. an infusion pump or bag) and further comprises a coupling device according to the present invention, the coupling device May constitute the “weak link(s)” of the tube(s) where the tube(s) may be “cut off” as a result of a pulling of the tube(s).

The coupling system is especially advantageous in case it is used in connection with an element inserted into a patient for a transfer of fluid to and/or from the patient. This is realized as a displacement of the element may not only be painful for the patient, but may also lead to consequences of the patient's treatment if the transferring of fluid due to the element's displacement is not performed correctly. By the coupling system of the present invention, it will be appreciated that equipment connected to the tubes such as (infusion) elements, pumps and/or bags, etc., may be spared from damage caused by a pulling of the tubes.

The coupling system of the present invention is further advantageous in that consequences related to medical staff and/or patients tripping and/or falling over (a) tube(s) connected via the coupling device may be mitigated.

The coupling system of the present invention is further advantageous in that its components (e.g. the first and second sealing elements) are designed to have relatively smooth outer surfaces, such that they may be cleaned and/or disinfected in an easy and efficient manner. For example, after a cleaning and/or disinfection of a dissembled coupling device, the respective components of the coupling device may subsequently be reassembled into the coupling device.

The coupling system of the present invention is further advantageous in that it is relatively inexpensive to manufacture and is easily assembled. Consequently, the coupling system may primarily be designed for single use, i.e. the coupling system may for example be used for one patient and one therapy (e.g. infusion).

The coupling device of the coupling system of present invention is further advantageous in that its design minimizes dead space of the fluid path through the coupling device, thereby mitigating the occurrence of infectious agents. Furthermore, the inventive design of the coupling device mitigates leakage of the fluid.

By the wording “at least one second tube arranged to transfer a respective second fluid”, it is here meant that in case of a plurality of second tubes (e.g. a (first) second tube, a (second) second tube, a (third) second tube, etc.), each second tube is arranged to transfer a respective second fluid (e.g. a (first) second fluid, a (second) second fluid, a (third) second fluid, etc.

According to an embodiment of the present invention, there is provided a system for transferring fluids into a patient, wherein the system comprises the coupling system according to any one of the preceding embodiments. The system comprises a first container arranged to contain a first fluid, wherein the first tube is connected to the first container by a first end portion of the first tube. The system further comprises an element connected to a second end portion of the first tube, for insertion into the patient, for allowing a transfer of the first fluid from the first container into the patient via the first tube. The system further comprises a second container arranged to contain a second fluid, wherein a second tube of the at least one second tube is connected to the second container by a first end portion of the second tube, wherein the first connector element and the second connector element of the coupling device are configured to matingly connect for a transfer of the second fluid from the second container into the patient via the second tube and the first tube.

By the wording “a second tube of the at least one second tube is connected to the second container”, it is here meant that one of the at least one second tube (i.e. a single second tube of the second tube(s)) is connected to the second container.

It will be appreciated that the (medical) element may be an element arranged for insertion into a patient and configured to transfer a fluid into, or out from, a patient, such as a needle, cannula, catheter, trocar, or the like. The present embodiment is advantageous in that it may increase the safety during a medical process, e.g. an infusion process.

Further objectives of, features of, and advantages with, the present invention will become apparent when studying the following detailed disclosure, the drawings and the appended claims. Those skilled in the art will realize that different features of the present invention can be combined to create embodiments other than those described in the following.

### BRIEF DESCRIPTION OF THE DRAWINGS

This and other aspects of the present invention will now be described in more detail, with reference to the appended drawings showing embodiment(s) of the invention.

FIGS. 1 and 2 are schematic views of infusion arrangements according to the prior art,

FIG. 3a is a schematic view of a coupling system according to an exemplifying embodiment of the present invention,

FIG. 3b is a schematic view of a portion of a coupling device of a coupling system according to an exemplifying embodiment of the present invention,

FIG. 4 is a schematic view of a coupling system according to an exemplifying embodiment of the present invention, and

FIG. 5 is a schematic view of a coupling device of a coupling system according to an exemplifying embodiment of the present invention.

### DETAILED DESCRIPTION

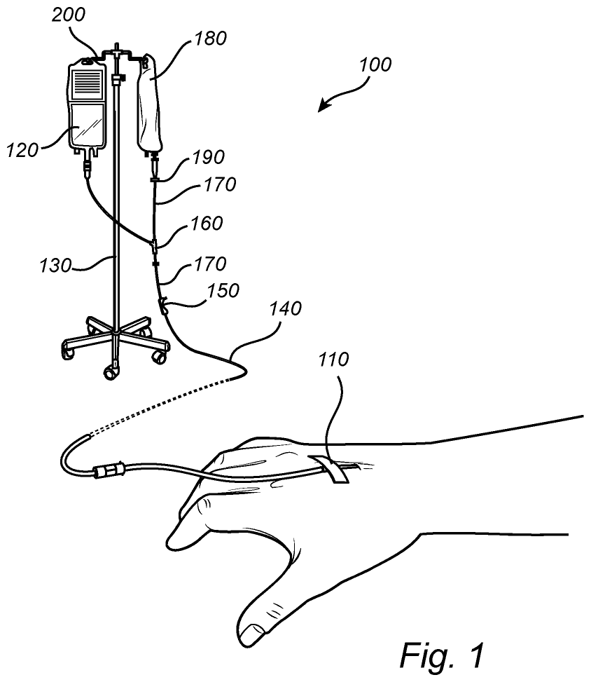

FIG. 1 is a schematic view of an infusion arrangement 100 according to the prior art. The infusion arrangement comprises a venous access device, VAD, 110 which, in turn, comprises a needle, cannula, catheter, trocar, or the like, for infusion into a patient, e.g. via the patient's hand. The infusion arrangement 100 comprises a first (primary) reservoir or container 120 in form of, for example, a (plastic) bag, which may contain a primary fluid such as a saline solution or a dextrose solution. The first reservoir 120 is suspended from a rack or stand 130 situated higher than the VAD 110, thereby allowing the primary fluid to enter the patient's bloodstream by the force of gravity via a primary tube 140 connected to the VAD 110. A clamp 150 is arranged along the primary tube 140, wherein the clamp 150 can be used to close off the flow of fluid through the primary tube 140 from any point above the clamp 150. Above the clamp 150 is a Y-port 160 which, via a secondary tube 170, is connected to a secondary reservoir or container 180 in form of a (“piggyback”) bag. A spike fitting (not shown) is inserted into the reservoir 180 by puncturing it, to allow the fluid inside the reservoir 180, such as drugs and/or medicine, to flow into the patient when the clamp 150, and a clamp 190 arranged along the secondary tube 170, are open. The second reservoir 180 is suspended from the same rack or stand 130 for allowing the medication (secondary fluid) to enter the patient's bloodstream. For administration of the secondary fluid, the first reservoir 120 must be positioned below the second (“piggyback”) reservoir 180, and the rack or stand 130 may comprise an extension hook 200 for this purpose. After administration of the medication(s)/drug(s), the primary fluid is allowed to flow through the primary tube 140 such that all medication/drug content is provided to the patient.

A problem with the infusion arrangement 100 as described is that upon usage of the secondary tube 170, the primary tube 140 and the Y-port 160, needleless connectors (NCs) are often used. As there are various different designs of the NCs, this make it difficult for medical staff to identify the features which present the least risk and greatest safety. Furthermore, the designated clamping-disconnection sequence of the infusion arrangement 100 as described is of vital importance, as this is required for the NCs to work as designed. More specifically, NCs with a negative fluid displacement should be clamped before disconnection and positive fluid displacement NCs clamped after disconnection (as clamping is required to prevent air embolism), leading to a complicated operation. Yet another problem of the NCs is related to the risk of an insufficient sealing, which consequently may lead to leakage. It should be noted that this may be especially hazardous if the infusion liquid is toxic. Furthermore, the provision of CSTD devices in the infusion arrangement 100 may be problematic, as the efficacy of these depend on their designs. Hence, the infusion arrangement 100 as disclosed, and the operation thereof, is complex and complicated, which may be dangerous for the medical staff and/or the patient.

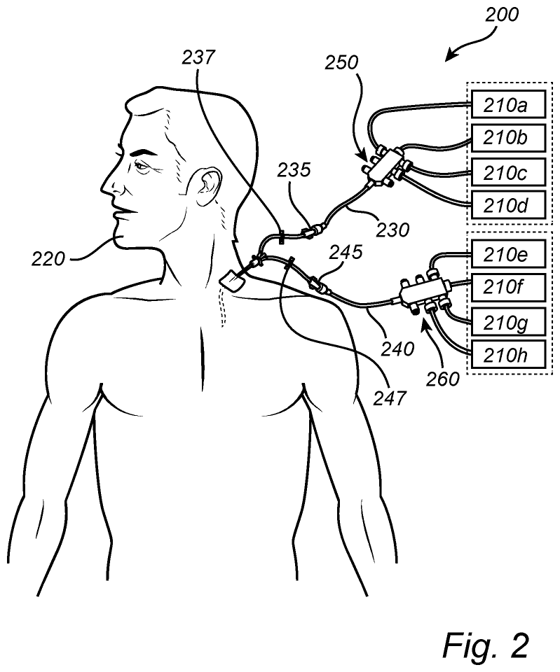

FIG. 2 is a schematic view of an infusion arrangement 200 according to the prior art. It should be noted that the infusion arrangement 200 of FIG. 2 may have the same or similar principles and/or features to the infusion arrangement 100 as exemplified in FIG. 1, and it referred to FIG. 1 and the associated text for an increased understanding. In FIG. 2, the infusion arrangement 200 comprises a plurality of infusion containers 210a-h, the fluids of which are to be administrated into a patient 220 via a (first) tube 230, comprising a (first) access port 235 and a clamp 237, and via a (second) tube 240, comprising a (second) access port 245 and a clamp 247. A first multiport connector 250 is provided between the tube 230 and infusion containers 210a-d, and analogously, a second multiport connector 260 is provided between the tube 240 and infusion containers 210e-h. The infusion arrangement 200 in FIG. 2 suffers from similar or the same drawbacks, limitations and problems as the infusion arrangement 100 in FIG. 1 as the arrangement, as well as its operation, is complex. Notably, by the provision of NCs, clamps and/or CSTD devices in/on the tubes 230, 240, on the first and/or second access ports 235, 245, and/or the first and/or secondary multiport connector(s) 250, 260, the infusion arrangement 200 is complex and complicated. Consequently, the operation of the infusion arrangement is cumbersome, and may furthermore be hazardous to the medical staff and/or the patient.

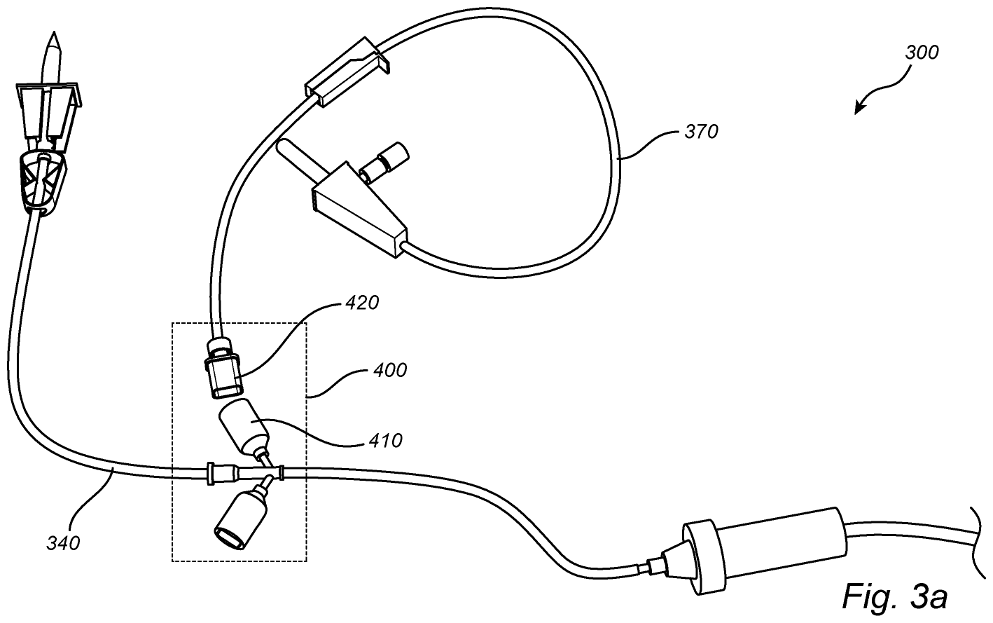

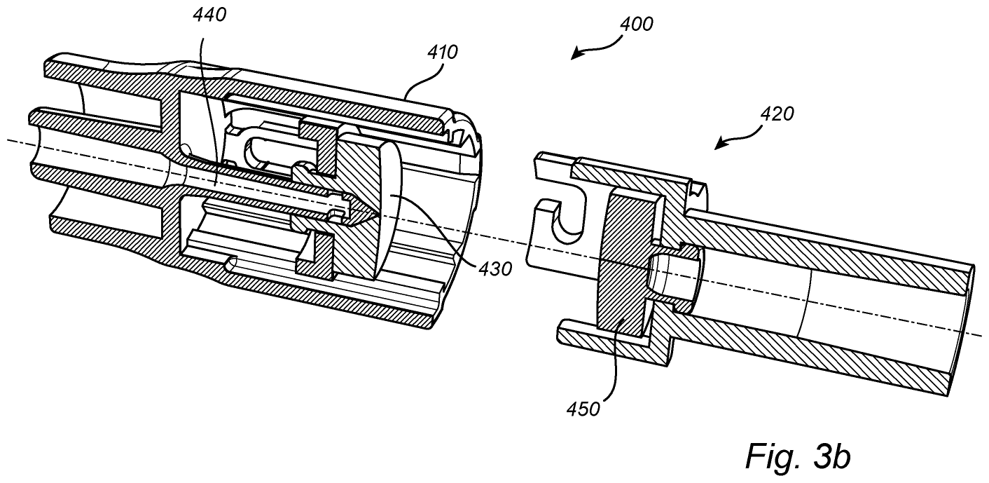

FIG. 3a is a schematic view of a coupling system 300 according to an exemplifying embodiment of the present invention. The coupling system 300 is primarily intended for a medical arrangement configured to transfer fluids into a patient. The coupling system 300 comprises a first tube 340, wherein the first tube 340 may be arranged to transfer a first fluid. The coupling system 300 further comprises at least one second tube 370. Each second tube of the at least one second tube 370 may be arranged to transfer a respective second fluid. FIG. 3a only discloses a single second tube 370, but it should be noted that the coupling system 300 may comprise substantially any number of second tubes 370. The coupling system 300 further comprises a coupling device 400 configured for a connection of the at least one second tube 370 with the first tube 340. The coupling device 400 comprises at least one first connector element 410 and at least one second connector element 420. It will be appreciated that the manifold structure of the coupling device 400 may comprise substantially any number of first and second connector elements 410, 420. The respective first and second connector elements 410, 420 are configured to matingly connect, e.g. for a transfer of the respective second fluid from the at least one second tube 370 to the first tube 340.

FIG. 3b is a schematic view of a portion of a coupling device 400 of a coupling system according to an exemplifying embodiment of the present invention. The first connector element 410 comprises a first sealing element 430 and a tube portion 440. The second connector element 420 comprises a second sealing element 450. In a non-connected state of the coupling device 400, as shown in FIG. 3b, the first sealing element 430 and the second sealing element 450 seal the second tube 370 from the first tube 340. In a connected state of the coupling device 400, the tube portion 440 projects through the first and second sealing elements 430, 450, for enabling a transfer of fluid through the coupling device 400.

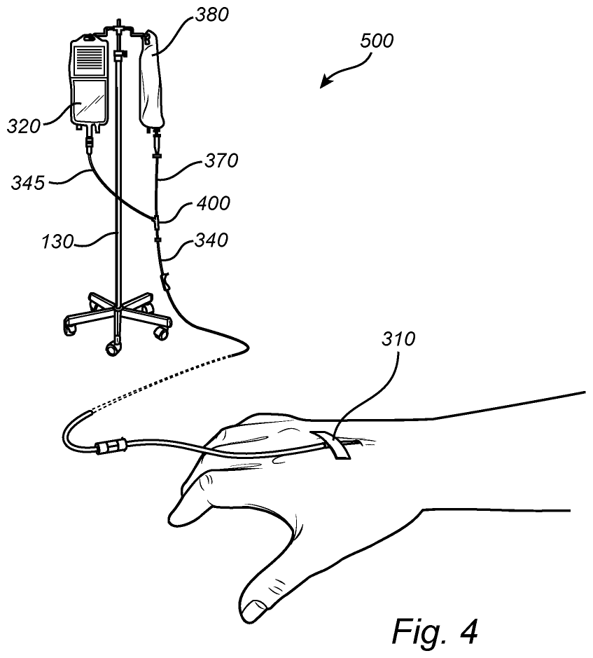

FIG. 4 is a schematic view of a coupling system 500 according to an exemplifying embodiment of the present invention. The coupling system 500 comprises an element 310 such as e.g. a needle, cannula, catheter, trocar, or the like, for infusion into a patient, e.g. via the patient's hand. The coupling system 500 comprises a first (primary) container 320 arranged to contain a first (primary) fluid, such as a saline solution or a dextrose solution. The first container 320 is suspended from a rack or stand 130 situated higher than the element 310, thereby allowing the primary fluid to enter the patient's bloodstream by the force of gravity via a first tube 340 connected to the element 310. The first tube 340 is connected to the first container 320 by a first end portion of a branch 345 of the first tube 340. Hence, by the element 310, connected to a second end portion of the first tube 340, a transfer of the first fluid is enabled from the first container 320 into the patient via the first tube 340. A second container 380 in form of a (“piggyback”) bag is arranged to contain a second fluid such as drugs and/or medicine. A second tube 370 is connected to the second container 380 by a first end portion of the second tube 370. The second container 380 is suspended from the same rack or stand 130 for allowing the medication (secondary fluid) to enter the patient's bloodstream. For administration of the secondary fluid, the first container 320 must be positioned below the second (“piggyback”) container 380, and the rack or stand 130 may comprise an extension hook for this purpose. After administration of the medication(s)/drug(s), the primary fluid is allowed to flow through the primary tube 340 such that all medication/drug content is provided to the patient. The coupling system 500 further comprises a coupling device 400 as described in FIG. 3a and/or FIG. 3b, and it is referred to these figures and the associated text for an understanding of the features and operation of the coupling device 400.

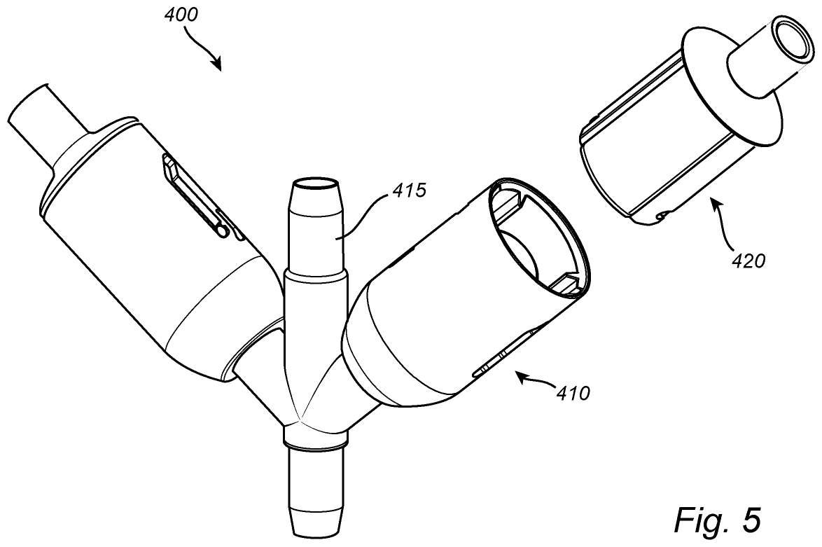

FIG. 5 is a schematic view of a portion of a coupling device 400 of a coupling system according to an exemplifying embodiment of the present invention. The first connector element 410 and the second connector element 420 of the coupling device 400 are configured to matingly connect. The coupling device 400 further comprises an integrated portion 415 connectable to the first tube (not shown).

The person skilled in the art realizes that the present invention by no means is limited to the preferred embodiments described above. On the contrary, many modifications and variations are possible within the scope of the appended claims. For example, it will be appreciated that the figures are merely schematic views of coupling systems according to embodiments of the present invention. Hence, any elements/components of the coupling systems and/or coupling device thereof may have different dimensions, shapes and/or sizes than those depicted and/or described.

1-2. (canceled)

3. A system for transferring fluids into a patient, wherein the system comprises:
a coupling system for fluid transfer, the coupling system comprising
a first tube arranged to transfer a first fluid,
a plurality of second tubes arranged to transfer a respective second fluid,
a coupling device configured for a connection of the plurality of second tubes with the first tube, wherein the coupling device comprises
a manifold structure comprising at least two first connector elements and at least two second connector elements configured to matingly connect for a connection between the plurality of second tubes and the first tube for a transfer of the respective second fluid from the plurality of second tubes to the first tube, wherein each first connector element comprises a first sealing element and a tube portion, and wherein each second connector element comprises a second sealing element,
whereby, in a non-connected state of the coupling device, the first sealing element and the second sealing element seal the respective second tube from the first tube, and
whereby, in a connected state of the coupling device, the tube portion projects through the first and second sealing elements, for fluid connection between the tube portion of the first connector element and the second connector element for enabling a transfer of fluid through the coupling device,
a first container arranged to contain a first fluid, wherein the first tube is connected to the first container by a first end portion of the first tube,
an element, connected to a second end portion of the first tube, for insertion into the patient, for allowing a transfer of the first fluid from the first container into the patient via the first tube,
a second container arranged to contain a second fluid, wherein a second tube of the plurality of second tubes is connected to the second container by a first end portion of the second tube,
wherein the first connector element and the second connector element of the coupling device are configured to matingly connect for a transfer of the second fluid from the second container into the patient via the second tube and the first tube.
